(19) United States
(12) Patent Application Publication
(10) Pub. No.: US 2016/0146761 A1
(43) Pub. Date: May 26, 2016

(54) GRADED STRUCTURE FILMS

(71) Applicant: EMPIRE TECHNOLOGY DEVELOPMENT LLC, Wilmington, DE (US)

(72) Inventor: Yasuhisa FUJII, Kyoto (JP)

(21) Appl. No.: 14/897,216

(22) PCT Filed: Jun. 10, 2013
(86) PCT No.: PCT/US13/44993

§ 371 Date: Dec. 9, 2015

(51) Int. Cl. G01N 29/036; C23C 14/48; G01N 33/00; G01N 29/02

(52) U.S. Cl. CPC G01N 29/036; G01N 29/022; C23C 14/48; G01N 33/0044; G01N 33/004; G01N 33/0062; G01N 2291/014

(57) ABSTRACT

Devices, films, and methods for the detection of target molecules are provided. The devices, films and methods can include sensitive films and a vibration detecting unit. The vibration detecting unit can be a convex or an inverse mesa vibration detecting unit.

### BACKGROUND

A variety of devices and methods exist for sensing chemicals in the environment. In some situations, the methods and/or devices employ various films for the physical aspect of the detection in these sensing devices. Such films can be created in a number of ways, such as ink jet printing, dispensing, spin coating, dipping, etc.

### SUMMARY

In some embodiments, methods and devices are provided for detecting the presence or absence of molecules in the environment.

In some embodiments, a method of making a graded chemical sensor is provided. The method can include providing at least a first vibration detecting unit, providing a sensitive film over the first vibration detecting unit, and differentially implanting a first ion concentration in a first portion of the sensitive film relative to a second portion of the sensitive film, thereby providing a graded sensitive film for a graded chemical sensor.

In some embodiments, a method of making a graded chemical sensor is provided. The method can include providing at least a first vibration detecting unit, providing a sensitive film over vibration detecting unit, and differentially heating a first portion of the sensitive film relative to a second portion of the sensitive film, thereby providing a graded sensitive film for a graded chemical sensor.

In some embodiments, a graded chemical sensor is provided. The sensor can include a substrate, at least one vibration detecting unit, and a graded sensitive film over the substrate. The graded sensitive film can be produced by differentially implanting a first ion concentration in a first portion of the sensitive film relative to a second portion of the sensitive film, thereby providing a graded sensitive film for a graded chemical sensor.

In some embodiments, a graded chemical sensor is provided. The graded chemical sensor can include a substrate, at least one vibration detecting unit, and a graded sensitive film over both at least part of the substrate and the at least one vibration detecting unit, the graded sensitive film being produced by differentially heat treating a first portion of the sensitive film relative to a second portion of the sensitive film.

In some embodiments, a graded chemical sensor is provided. The graded chemical sensor can include a substrate, a first vibration detecting unit on the substrate, and a graded sensitive film over both at least a part of the substrate and the first vibration detecting unit. The graded sensitive film can include a first portion that includes a first ion concentration and a second portion that includes a second ion concentration. The second ion concentration is different than the first ion concentration.

In some embodiments, a graded chemical sensor is provided. The graded chemical sensor can include a substrate, a first vibration detecting unit, and a graded sensitive film over both at least a part of the substrate and the first vibration detecting unit. The graded sensitive film can include a first portion that includes a first set of characteristics that are a same as a portion of a sensitive film that has been heat treated to a first amount and a second portion that includes a second set of characteristics that are a same as a portion of a sensitive film that has been heat treated to a second amount. The first amount and the second amount can be different.

In some embodiments, a method of detecting a presence or absence of a target is provided. The method can include providing a graded chemical sensor, the graded chemical sensor can include a substrate, a first vibration detecting unit on the substrate, and at least one of: a) a graded sensitive film over both at least a part of the substrate and the first vibration detecting unit. The graded sensitive film includes a first portion that includes a first ion concentration and a second portion that includes a second ion concentration. The second ion concentration is different than the first ion concentration. Alternatively, b) a graded sensitive film over both at least a part of the substrate and the first vibration detecting unit. The graded sensitive film includes a first portion that includes a first set of characteristics that are a same as a portion of a sensitive film that has been heat treated to a first amount and a second portion that includes a second set of characteristics that are a same as a portion of a sensitive film that has been heat treated to a second amount. The first amount and the second amount can be different. The method can further include contacting a sample to the graded sensitive film. If the sample includes the target, the target associates with the sensitive film and changes the vibrational frequency of the sensitive film. The method can further include detecting whether the vibrational frequency of the sensitive film changes.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1A is a drawing depicting some embodiments of a convex vibration detecting unit.

FIG. 1B is a drawing depicting some embodiments of an array of inverse mesa vibration detecting units.

FIG. 2 is a flowchart depicting some embodiments of a method for detecting the presence or absence of a target using the sensor device provided herein.

FIG. 3A is a flow chart depicting some embodiments of a method of making a film.

FIG. 3B is a drawing depicting a method of treatment of a film.

FIG. 3C is a drawing depicting a resulting film having a five by five array.

FIG. 3D is a drawing depicting some embodiments of a sensor device.

FIG. 4A is a drawing depicting some embodiments of a front of a QCM substrate.

FIG. 4B is a drawing depicting some embodiments of a back of a QCM substrate.

FIG. 4C is a drawing depicting some embodiments of a graded sensitive film.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Provided herein are sensitive films which have been selectively and/or differentially treated. The treatment parameters include heat-treatment and/or ion implantation treatment. These treatments result in physical changes to the sensitive films, which in some embodiments results in altering the physical characteristics of the sensitive films. Thus, rather than dealing with the difficulties of altering a sensitive film during the creation of the sensitive film itself, these treatments allow for the creation of further varied sensitive films by an additional process which can occur during or after the creation of the sensitive film itself. In some embodiments, these treated sensitive films can be placed on a convex vibration detection unit and/or an inverse mesa detection unit. In some embodiments, as these treated films can have localized, differing, physical properties, the resulting films exhibit a wide variety of sensitivity properties. In some embodiments, this allows for a chemical sensor array with a large number of detection abilities (for example, various sensitivities to various compounds) over a single quartz substrate.

In some embodiments, the treatment parameter is a heat-treatment. For example, a sensitive film is deposited onto the vibration-detecting unit (for example, onto a substrate). During or after this film deposition, a combinatorial laser heating technique (for example) can be used to heat the sensitive film locally in a non-contact manner under laser irradiation conditions that differ over various portions of the film, thereby producing a graded (or differentiated) synthetic film having various portions that can have different heat-treatment histories from one another. The heat-treatment alters the sensitive film (for example, it can promote a reaction in the treated area and/or simply allow for atomic reordering that leads to a stable structure or compound), consequently allowing the sensitive film on the substrate to be endowed with a variety of physical properties, despite the initial state of the sensitive film.

In some embodiments, the treatment parameter can be ion implantation. For example, a sensitive film is deposited onto the vibration-detecting unit. Different portions of the film are then exposed to different types and/or concentrations of ions. In some embodiments, the ions can be implanted via a combinatorial ion implantation technique to produce ion-implanted graded film, which is then (optionally) subjected to heat treatment. In some embodiments, according to the ion implantation conditions, lattice defects can be introduced and non-equilibrium reactions can be promoted through the interaction between the crystal lattice and the high-energy ion beam, thereby altering the structure of the film. Other alterations can also be employed. The result of the treatment is that the initial sensitive film is altered so that it can be endowed with a wide variety of physical properties.

With the first and second treatment options, it is possible to control the physical properties of the films, including at least one of more of the polarity, dielectric constant, solubility parameter, hydrophilicity, hydrophobicity, electrical charge, conductivity, free surface energy, magnetization, magnetic permeability, pH, etc.

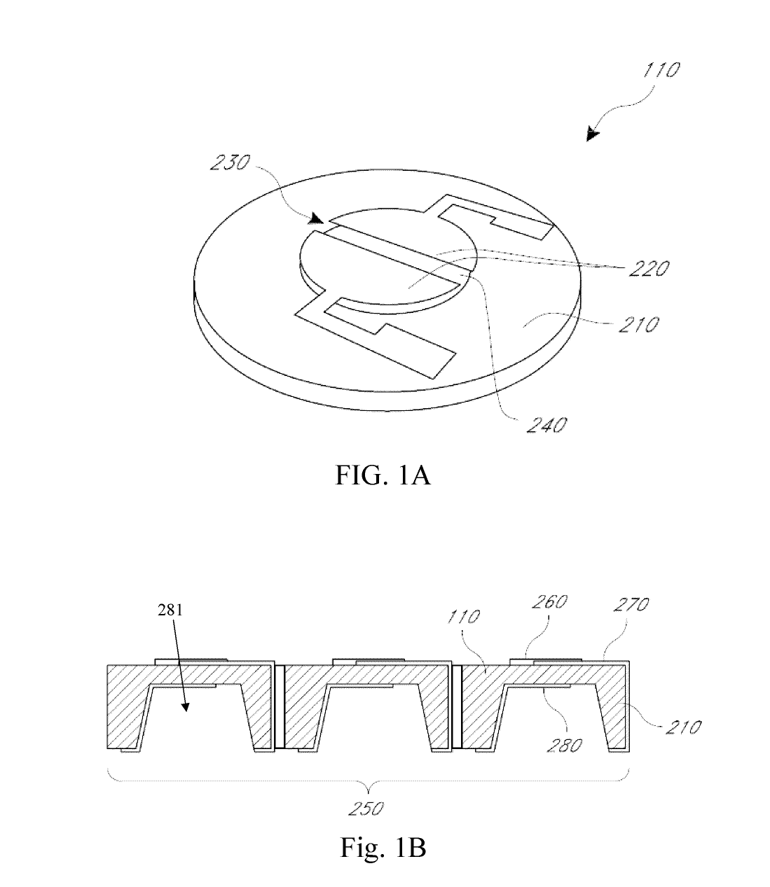

FIG. 1A depicts some embodiments of a vibration detecting unit 110 having a convex shape. The support 210 of the vibration detecting unit can be made of a variety of materials. The vibration detecting unit can include one or more excitation electrodes 220, on the support, which, through the application of an electrical potential, can establish the basal vibrational frequency of the system when in use. When these electrodes 220 are on the same side, there can be a gap 230 between them. In some embodiments, there can be a conductive film 240 that can be on the opposite side of the image shown in FIG. 1A. In some embodiments, the vibration detecting unit is an integral part of a substrate. For example, the vibration detection unit can be made from the substrate or be a part of the substrate.

In some embodiments, the convex shape of the vibration detecting unit can include one or more surfaces that are curved. In some embodiments, the convex shape can include one or more surfaces that are rounded in an outward direction. In some embodiments, the one or more surfaces can include a raised portion to effectively provide the convex shape.

In some embodiments, the convex vibration detecting unit can be a quartz crystal microbalance (QCM). In some embodiments, the convex vibration detecting unit can be a plano-convex QCM. In some embodiments, the convex vibration detecting unit can be a bi-convex QCM.

In some embodiments, the support for the convex vibration detecting unit can include AT-cut quartz crystal. In some embodiments, the convex vibration detecting unit can be miniaturized.

While two electrodes 220 are shown on the same side of the support 210 in FIG. 1A, in some embodiments, each side of the support 210 can have one of the electrodes, thereby removing any need for a gap 230.

As noted above, in some embodiments, the vibration detecting unit can be in an inverse-mesa shape. FIG. 1B depicts some embodiments of an array 250 of vibration detecting units 110 that include an inverse mesa shape. One or more electrodes 260, 270, and 280 can be associated with the support of the vibration detecting units 110. In some embodiments, the excitation electrodes 270 and 280 can be located on opposite sides of the support 210. In some embodiments, a conductive layer 260 can be positioned as part of the vibration detection unit.

In some embodiments, the inverse mesa shape can include a groove 281 within the substrate (and thus, be made of the substrate). In some embodiments, the groove can form a section of relative thinness in the substrate. In some embodiments, the inverse mesa (or walls of the groove) is made from the quartz substrate by etching a groove into the substrate. Thus, in some embodiments, the quartz substrate will be thinner in some sections relative to others. In some embodiments, electrodes can be formed on both sides of the thinner section of the inverse mesa. In some embodiments, the electrode portion of the thin crystal can be part of the sensor, with one or more additional graded layers being positioned over this thinner section.

The vibration detecting unit can include a support 210. In some embodiments, the support can be the same structure as the substrate on which the vibration detecting unit is positioned. Thus, in some embodiments, the vibration detecting unit is integral to the substrate. In some embodiments, the support 210 of the vibration detecting unit can be different and/or separate from the substrate.

In some embodiments, the vibration detecting unit can be formed on top of the substrate. In some embodiments, the substrate can have any suitable shape. The substrate can have a regular shape or an irregular shape. In some embodiments, the substrate can be a triangle, square, pentagon, hexagon, octagon, circle, oval, etc.

In some embodiments, the support has a thickness of 0.1 micron or more, for example, 0.1, 1, 10, 20, 30, 40, 50, 60, 70, 80, 90, 100, 1000, 10,000 microns or more, including any range between any two of the preceding values and any range above any one of the preceding values.

In some embodiments, the support can be made of any material capable of experiencing a frequency of oscillation. In some embodiments, the support can experience a piezoelectric effect. In some embodiments, the support can be made of quartz. In some embodiments, the support is substantially all quartz. In some embodiments, the support can include, as at least part of the piezoelectric element, lithium niobe oxide, zinc oxide, aluminum nitrid, lead zirconate titanate, etc.

In some embodiments, the vibration detecting unit can also include one or more conductive layers 220, 260, 270, 280. In some embodiments, the conductive layer can be made of any conductive material. In some embodiments, the conductive layer includes gold, copper, silver, aluminum, molybdenum, chromium, indium tin oxide, etc.

The conductive layer can be located on one or more surfaces of the support. In some embodiments, the conductive layer is on a top surface of the support. In some embodiments, the conductive layer is on a bottom surface of the support. In some embodiments, the conductive layer is on the top and bottom surface of the support.

In some embodiments, the conductive layer serves as at least one electrode. In some embodiments, the conductive layer includes two or more electrodes. In some embodiments, the electrode can be an excitation electrode to generate the basal level of vibration in the support and/or quartz. In some embodiments, the excitation electrode can include a separated electrode that has an electrode gap 230 between the two parts of the electrode. In some embodiments, the excitation electrode is a non-separated electrode. In some embodiments, a conductive film is on a top surface of the support and an excitation electrode is on a bottom surface of the support. In some embodiments, a conductive film is on a bottom surface of the support and an excitation electrode is on a top surface of the support. In some embodiments, the two separate electrodes are located on the same side of the vibration detecting unit. In some embodiments, the two separate electrodes are located on a bottom surface of the support.

In some embodiments, the sensor device can include two or more vibration detecting units. In some embodiments, the sensor device can include a plurality of vibration detecting units. For example, a plurality of vibration detecting units can be arranged in an array. The vibration detecting units can be arranged in any suitable configuration. In some embodiments, the vibration detecting units are spaced equal distance from one another. In some embodiments, the arrangement of the plurality of vibration detecting units is arbitrary and/or random. In some embodiments, the vibration detecting units form one or more arrays of vibration detecting units, such as 250. In some embodiments, the vibration detecting units are spaced apart as a function of the gradient change in the sensitive film. Thus, for example, the vibration detecting units are spaced so that meaningful changes in the amount of material of the sensitive film can be detected by a proximal vibration detecting unit. In some embodiments, the vibration detecting units are positioned so that fine changes can be observed. In some embodiments, the vibration detecting units are positioned so that a majority of the film above a vibration detecting unit is for detecting a single molecule species. Thus, a change in signal for the vibration detecting unit will indicate the presence of the target species that is absorbed by the film above the vibration detecting unit. In some embodiments, the vibration detecting units are positioned under sections of gradients of the film, such that a single vibration detecting unit can detect absorption in two or more gradient films (for example, 2, 3, 4, 5, 6, 7, 8, 9, 10 or more films). In such arrangements, the patterns of signals from the array can indicate what molecule species are binding to the film, as a single change in signal from a vibration detecting unit can indicate the presence (or absence) of any molecule that can absorb to the stack of sensitive film above it.

In some embodiments, vibration detecting units can be spaced 10⁻⁹, 10⁻⁸, 10⁻⁷, 10⁻⁶, 10⁻⁵, 10⁻⁴, 10⁻³, 10⁻², 10⁻¹, or 1 meter apart from one another, including any range between any two of the preceding values and any range above any one of the preceding values.

In some embodiments, the top surface of the first vibration detecting unit is in a same plane as the top surface of an adjacent vibration detecting unit. In some embodiments, substantially all of the surfaces of the vibration detecting units are in approximately the same plane.

In some embodiments, the array can include all the same type of vibration detecting units, for example, all convex vibration detecting units or all inverse mesa vibration detecting units. In some embodiments, the array can include convex vibration detecting units and inverse mesa vibration detecting units.

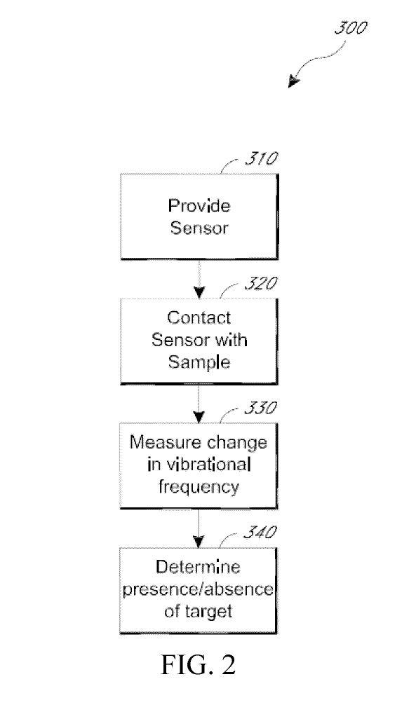

FIG. 2 depicts some embodiments of a method (300) for detecting the presence or absence of a target using the sensor devices provided herein.

The various devices and components provided herein can be employed for a variety of methods. In some embodiments, the method of detecting a presence or an absence of a target includes providing a sensor (block 310). In some embodiments, the sensor can include a sensitive film and a vibration detecting unit. The method can include contacting the sensor with a sample (block 320), which can be achieved in any number of ways, for example, flowing a sample that may include a target over a surface of the sensor. In some embodiments, the method includes measuring a change in vibrational frequency (block 330). In some embodiments, this measurement can be achieved by applying an electrical charge to the excitation electrode while the sensor is in a vacuum, and measuring a baseline vibrational frequency in the absence of a target. In some embodiments, a background environment can be taken into account, and thus, an initial baseline vibrational frequency is determined in an operating environment (or when the sensitive film is under standardized or “control” conditions). One can then determine the presence or absence of a target in the sample (block 340). This can be achieved by detecting any change in vibrational frequency. For example, a decrease in vibrational frequency from the baseline vibrational frequency can indicate an increase in mass, and thus, an increase in binding of a target molecule in the sample to the sensitive film, which is indicative of the presence and/or increase of the target molecule. Similarly, an increase in vibrational frequency from an earlier measured vibrational frequency can indicate a decrease in mass, and thus, a decrease in binding of the target molecule in the sample to the sensitive film, which is indicative of a decrease in amount of the target molecule in the sample. Also, a measured vibrational frequency that is substantially the same as the baseline vibrational frequency can indicate an unchanged mass, and thus, an absence of the target molecule in the sample. As noted above, an array of vibration detecting units can be used to detect more than one target and/or detect various concentrations of a target molecule.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The sensor device can be used in any suitable environment. In some embodiments, the sensor device can be used under vacuum. In some embodiments, the method and/or device can be employed with a fluid, such as a gas or a liquid.

In some embodiments, a sample can be provided to the sensor device by bringing the sample to a surface of the sensitive film. In some embodiments, the sample is flowed across a surface of the sensitive film. In some embodiments, the sample is placed on the sensitive film, allowed to sit and then removed. In some embodiments, a brief washing process can be performed between the application of the sample to the surface of the sensitive film and the measuring of a change in vibrational frequency. This can reduce any effect of nonspecific binding of the target molecule to the sensitive film.

In some embodiments, the change in vibrational frequency may be determined while a sample is being moved across a surface of the sensitive film. In such arrangements, the background vibrational frequency can take into account the impact of the sample presence and/or movement on the vibrational frequency. In some embodiments, the change in vibrational frequency may be determined when there is no sample movement across a surface of the sensitive film. In some embodiments, the change in vibrational frequency may be determined when there is no sample on a surface of the sensitive film, for example, when the sample has been removed and any target detected is that which remains after the removal of the bulk sample. Given the varied detection abilities of the treated sensitive films provided herein, in some embodiments, the vibrational frequency is expected to change across the surface of the sensitive film, as the sensing properties of the sensitive film change from one portion (that was subjected to one set of treatment conditions) to another portion (that was subjected to a second set of treatment conditions).

In some embodiments, the volume of the sample is at least 10⁻⁹, 10⁻⁸, 10⁻⁷, 10⁻⁶, 10⁻⁵, 10⁻⁴, 10⁻³, 10⁻², 10⁻¹, 1, 10, 10², 10³, 10⁴, 10⁵, 10⁶ liters or more, including any range above any one of the preceding values and any range between any two of the preceding values. In some embodiments, any flow rate can be used to apply the sample to the surface of the sensitive film. In some embodiments, the flow rate of the sample is at least 10⁻⁹, 10⁻⁸, 10⁻⁷, 10⁻⁶, 10⁻⁵, 10⁻⁴, 10⁻³, 10⁻², 10⁻¹, 1, 10, 10², 10³, 10⁴, 10⁵, 10⁶ liters/minute or more, including any range above any one of the preceding values and any range between any two of the preceding values.

In some embodiments, the sample can include one or more target. In some embodiments the target can be any material capable of interacting with the sensitive film. In some embodiments, the target can be a gas component. In some embodiments, the target can include at least one of ammonia (NH₃), hydrogen (H₂), hydrogen sulfide (H₂S), carbon monoxide (CO), and/or carbon dioxide (CO₂). In some embodiments, the target can include any component in a fluid-based diagnosis. In some embodiments, given that the treatment conditions can be employed to create a wide variety of treated portions on the surface, and that those differentially treated portions can have different sensitivities, the targets can be highly varied. As outlined below, in some embodiments, a desired target can be matched to a particularly treated sensitive film by treating the film with a wide variety of conditions (various heat and ion exposures for various durations) and passing the target over the film to identify what set of conditions modify the film adequately to be able to identify the target (by allowing the target to associate with the modified portion of the sensitive film).

In some embodiments, the sensitive film can be selected based on the target or targets that one desires to detect the presence and/or absence of. Thus, in some embodiments, any sensitive film can be used as long as it absorbs the target molecule. In some embodiments, the sensitive film directly absorbs the target molecule. In some embodiments, the sensitive film is associated with an agent that binds the target molecule. In some embodiments, the sensitive film selectively binds and/or absorbs the target molecule. In some embodiments, “selectively binds and/or absorbs the target molecule” can denote that the film absorbs more of the target and/or absorbs it more quickly and/or retains the target better than at least one other molecule species in a sample and/or in a standardized control sample. In some embodiments, any amount of superior binding and/or absorption is sufficient, for example, 1, 10, 100, 1000, 10,000, 100,000, or 1,000,000 percent better binding and/or absorption, including any range above any one of the preceding values and any range between any two of the preceding values.

In some embodiments, the sensitive film can include acrylic acid. In some embodiments, ammonia can associate with a film including acrylic acid, and thus, the film that includes acrylic acid can be used to detect ammonia. In some embodiments, the film can include palladium. In some embodiments, hydrogen can associate with a film including palladium, and thus, the film that includes palladium can be used to detect hydrogen. In some embodiments, the film can include zinc oxide. In some embodiments, hydrogen sulfide can associate with a film including zinc oxide. In some embodiments, the film can include titanium dioxide. In some embodiments, carbon dioxide can associate with a film including titanium dioxide. In some embodiments, the film includes TiO₂, ZrO₂, and/or WO₃, and any combination thereof. In some embodiments, any of the films presented herein can be treated as provided herein (for example a heat and/or ion implantation treatment) in whole or in portions.

The sensitive film can be made of any material suitable for associating with a target. In some embodiments, the material (or composition) of the sensitive film can be selected based on any number of parameters, for example, the polarity, dielectric constant, dissolution parameter, hydrophilicity, hydrophobicity, charge, and/or conductivity of the material. In some embodiments, these conditions can be adjusted via the treatment options provided herein (for example, heat and/or ion implantation). In some embodiments, the sensitive film selectively responds to a target.

In some embodiments, the sample includes two or more targets for example, 2, 3, 4, 5, 6, 7, 8, 9, 10, 20, 30 or more targets, including any range above any one of the preceding values and any range defined between any two of the preceding values. In some embodiments, the second target can be the same or substantially the same as the first target. In some embodiments, the second target can be different from the first target.

In some embodiments, the vibration detecting unit can measure a mass per unit area by measuring a change in frequency of the support and/or sensitive film. In some embodiments, the resonance can be altered by the addition or removal of a mass at or near a surface of the sensitive film. In some embodiments, changes in frequency can be determined to a high precision by measuring the mass densities down to a level of below 10,000 μg/cm², for example 10,000, 1000, 100, 10, 1, 0.1, 0.01, or 0.001 μg/cm² or lower, including any range below any one of the preceding values and any range between any two of the preceding values.

In some embodiments, the vibration detecting units can produce a basal standing wave via the application of an alternating current to the excitation electrodes of the support. This can induce oscillations in the form of a standing shear wave. In some embodiments, the vibration detecting unit can detect the basal standing wave. In some embodiments, the vibration detecting unit can detect a change in the basal standing wave.

In some embodiments, the vibration detecting unit can allow for a resolution of frequency of oscillation that is as low as 1 Hz on crystals with a fundamental resonant frequency in the 4-6 MHz range. The frequency of oscillation of the vibration detecting unit is partially dependent on the thickness of the support and/or the sensitive films over the support. Where the other influencing variables remain constant, a change in mass of the support and/or sensitive film, for example an increase in mass due to the binding of the target on the support and/or sensitive film, will correlate to a change in frequency.

In addition to measuring the frequency, in some embodiments, the dissipation can be measured to help analysis. The dissipation is a parameter quantifying damping in the sensor system, and is related to the sample's viscoelastic properties. The dissipation is equal to the ratio of bandwidth, and frequency of oscillation.

In some embodiments, this frequency change can be quantified and correlated to the change in mass of the support and/or sensitive film. In some embodiments, the presence of a target can be determined by a change (decrease) in the vibrational frequency. In some embodiments, the absence can be determined by a lack of change in vibrational frequency (or an increase in vibrational frequency).

Any of a number of various techniques can be used for measuring to quantify and/or correlate the mass change. In some embodiments, techniques can include, but are not limited to, Sauerbrey's equation, Ellipsometry, Surface Plasmon Resonance (SPR) Spectroscopy, and/or Dual Polarisation Interferometry.

In some embodiments, the change in frequency correlating to the amount of a target associated with a sensitive film can be solved by employing Equation I:

Δf=2f₀²m_{f}/A(ρ_{q}μ_{q})^{1/2} Equation I

Δf: Change in resonant frequency
f₀: Resonant frequency
ρ_{q}: support density (for example Quartz 2.65 g/cm³)
μ_{c}: Frequency constant 1.67*10⁵ cmHz
m_{f}: Change in mass due to association of target
A: Electrode area

### f₀ (MHz)=1670/t

t=thickness of support (μm)

In some embodiments, the relationship between an amount of target in a sample and the change in mass and/or change in frequency can be determined by correlating known controls, for example, samples with a known amount of one or more targets in the sample, with a specific change in mass and/or change in frequency. In some embodiments, the change in electrical signal from the vibration detecting unit can be correlated to a specific amount of a target and/or range of a target in a sample by comparing known controls with a specific change in electrical signal from the vibration detecting unit.

In some embodiments, the sensitive film can have a thickness of about 0.1 nm to about 1,000,000 nm. In some embodiments, the sensitive film has a thickness of about 1,000,000, 100,000, 10,000, 1,000, 100, 10, 1, or 0.1 nm, including any range above any one of the preceding values and any range defined between any two of the preceding values. In some embodiments, the sensitive film has a maximum thickness of about 5 nm.

The sensitive film can be configured to associate with the target molecule. In some embodiments, the sensitive film can absorb the target molecule, or at least associate sufficiently with the target molecule such that the mass of the sensitive film is altered.

In some embodiments, the sensitive film is located over the vibration detecting unit. In some embodiments, the sensitive film can be above, have a common end point, and/or have a common border with the vibration detecting unit. In some embodiments, the sensitive film is adjacent to the vibration detecting unit. In some embodiments, the sensitive film contacts the vibration detecting unit. In some embodiments, the sensitive film adjoins, is contiguous with, and/or is juxtaposed to the vibration detecting unit. In some embodiments, the sensitive film is in close proximity to but does not contact the vibration detecting unit. In some embodiments, the sensitive film has an interface with the vibration detecting unit and/or the conductive film.

In some embodiments, a sensitive film can be placed on the conductive layer. In some embodiments, the sensitive film can be a biomaterial. In some embodiments, antibodies, antigens, receptors, and/or ligands can be placed in and/or bonded to the sensitive film for further options for detection and/or binding.

In some embodiments, the sensitive film is placed on the vibration detecting unit directly or indirectly. In some embodiments, the sensitive film is placed over the conductive layer. In some embodiments, the sensitive film is placed over the substrate. In some embodiments, one or more intervening layers can be located between the sensitive film and the vibration detecting unit.

In some embodiments, a mass of the sensitive film is distributed substantially at a middle portion the vibration detecting unit. In some embodiments, the sensitive film is at least partially overlaying a portion of the vibration detecting unit. The sensitive film can be physically coupled to the vibration detecting unit such that changes in the mass of the film can be detected by the vibration detecting unit. In some embodiments, the treated portion of the sensitive film is distributed substantially at a middle portion the vibration detecting unit. In some embodiments, the treated portion of the sensitive film is at least partially overlaying a portion of the vibration detecting unit.

In some embodiments, the sensor device can include two or more sensitive films, for example, 2, 3, 4, 5, 6, 7, 8, 9, 10, 20, 30 or more films, including any range above any one of the preceding values and any range defined between any two of the preceding values. In some embodiments, the second sensitive film can have the same composition as the first sensitive film. In some embodiments, the second sensitive film can have a different composition from the first sensitive film. In some embodiments, the two or more sensitive films can alternate in composition. For example, in some embodiments, a subsequent sensitive film can have the same composition as the first sensitive film, while the second sensitive film can have a different composition than the first and the subsequent sensitive film.

In some embodiments, one or more of the sensitive films can be graded. In some embodiments, the first and second sensitive films can have the same or substantially the same gradient. In some embodiments, the first and second sensitive films can have different gradients. In some embodiments, the first and second sensitive films overlap at their graded areas. Such an overlap allows for numerous layers to be positioned in a smaller area, and still be monitored by the vibration detecting units. In some embodiments, the sensitive film can be graded according to the presence of various treated portions. In some embodiments, the sensitive film can be graded by layering various components of the sensitive film (which can subsequently also be treated as provided herein).

In some embodiments, the first sensitive film includes acrylic acid, the second sensitive film includes palladium, and the third sensitive film includes zinc oxide.

In some embodiments, at least a portion of each of the two or more sensitive films can overlap one another. In some embodiments, the two or more sensitive films substantially overlap one another. In some embodiments, the two or more sensitive films overlap one another in an area over a vibration detecting unit.

In some embodiments, the two or more overlapping sensitive films have a maximum thickness of about 1,000,000 nm or less, for example, 1,000,000, 100,000, 10,000, 1,000, 500, 200, 190, 180, 170, 160, 155, 150, 145, 140, 135, 130, 120, 100, 50, 25, 10, 5, or 1 nm, including any range below any one of the preceding values and any range defined between any two of the preceding values.

In some embodiments, each detection site can be monitored by a vibration detecting unit that sends an electrical signal for further processing. In some embodiments, the source of the signal (which vibration detecting unit the signal came from) is monitored and/or can be determined. In some embodiments, for example where it is not important which sensitive film had a change in mass due to a target, the source of the signal need not be monitored and/or recorded and/or determined. In some embodiments, each treated portion is associated with a specific vibration detecting unit.

In some embodiments, the sensor device can include two or more detection sites, for example, 2, 3, 4, 5, 6, 7, 8, 9, 10, 20, 30, 40, 50, 60, 70, 80, 90, 100 or more detection sites, including any range above any one of the preceding values and any range defined between any two of the preceding values. In some embodiments, the second detection site can be the same or substantially the same as the first detection site. In some embodiments, the second detection site can have the same sensitive film composition as the first detection site. In some embodiments, the second detection site can have a different sensitive film composition from the first detection site. In some embodiments, the second detection site can have the same type of vibration detecting unit as the first detection site. In some embodiments, the second detection site can have a different vibration detecting unit from the first detection site. In some embodiments, a detection site has been treated with a treatment as provided herein. In some embodiments, each detection site has a different treatment history. In some embodiments, a detection site can have one or more treated portions. In some embodiments, one or more detection sites can have the same or similar treatment histories.

In some embodiments, the two or more detection sites can be located on a graded composition film structure. As will be appreciated by one of skill in the art, given the present disclosure, by increasing the number of vibration detecting units in the substrate, it becomes possible to use a graded-composition film structure having a more finely graded composition. The vibration detecting units (or detection sites) can be in any suitable arrangement.

In some embodiments, the sensor device is configured to detect the presence or absence of each of two or more targets. In some embodiments, the second detection site is configured to detect the presence or absence of a second target (for example, by having a different treatment history from the first detection site). In some embodiments, a detection site will only bind and/or absorb one target molecule. In some embodiments, a detection site can bind and/or absorb more than one target molecule. In some embodiments, a detection site can bind and/or absorb a class of molecules selectively over other detection sites. As noted below, this ability can be further diversified by applying different treatment histories to the sensitive film over different detection sites.

As the distance between vibration detecting units decreases, negative effects due to interference between vibration detecting units can occur. As will be appreciated by one of skill in the art, given the present disclosure, the various vibration detection units provided herein can reduce interference between the vibration detecting units. In some embodiments, the application of a convex vibration detection unit allows for less interference between the vibration detection units in an array. In some embodiments, the application of an inverse mesa vibration detection unit allows for less interference between the vibration detection units in an array. With such configurations, it becomes possible to reduce and/or prevent interference between vibration detecting units in an array, including aspects such as propagation and/or reflection. In some embodiments, a convex vibration detecting unit has part of its vibrating mass distributed in a middle portion of the resonator, so that vibration energy is constrained closer to the middle portion of the unit. As such, there can be a greater interference prevention effect as this section is more isolated. Similarly, the additional structure of the inverse mesa arrangement allows for less interference between the various vibration detecting units. In some embodiments, when combined with the treatment histories provided herein, the ability to position the vibration detecting units closer together allows for various treated detection sites to be positioned more closely together. In some embodiments, when combined with the treatment histories provided herein, the ability to position the vibration detecting units closer together allows for a wider variety of detection sites, as areas across a treated portion can exhibit differential properties in some embodiments (for example, on the edges and/or deeper sections of a treated portion).

In some embodiments, convex vibration detecting units have raised portions on their surfaces. In some embodiments, inverse-mesa vibration detecting units have recesses on their surfaces. In some embodiments, the vibration detecting units are quartz crystal microbalances.

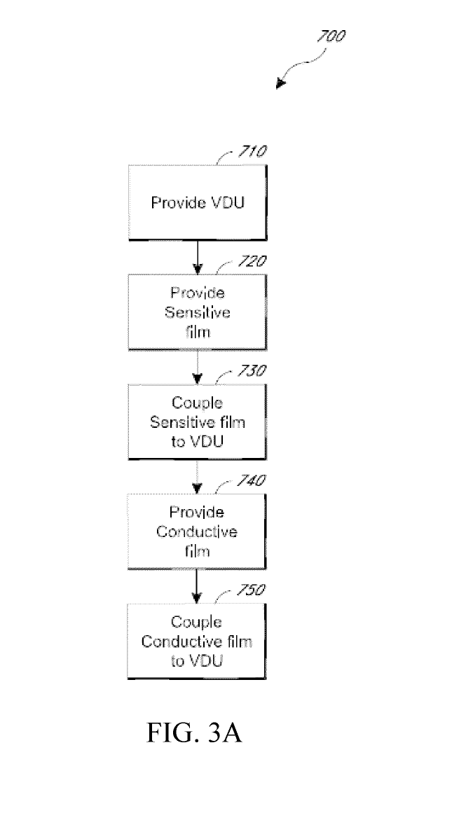

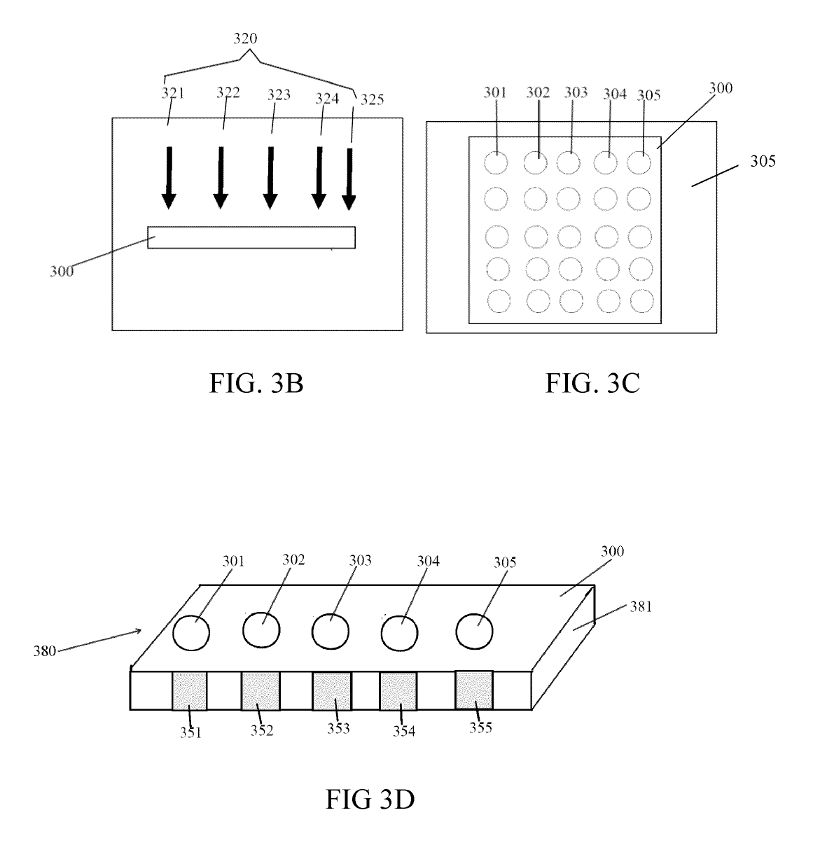

FIG. 3A depicts some embodiments of a method of manufacturing a sensor device including a graded sensitive film as described herein. In some embodiments, the method includes but is not limited to providing a vibration detecting unit (block 710) and providing a sensitive film (block 720). In some embodiments, one can couple the sensitive film to the vibration detecting unit (block 730). In some embodiments, coupling can be achieved by depositing the sensitive film onto the vibration detecting unit. The method can further include providing a conductive film (block 740). The conductive film is coupled to the vibration detecting unit (block 750). In some embodiments, the coupling can be achieved by depositing the conductive film onto the vibration detecting unit. As outlined below in more detail, these sensitive films can have various portions of them treated (for example, with heat and/or ion implantation) to provide greater diversity in their sensitivity and/or selectivity to target molecules.

As noted above, in some embodiments, the disclosed sensitive films can be treated to further differentiate the sensitive films. This allows for further diversity in the sensitive film, and thus, additional diversity in what, and the degree to which, various targets can be detected. As noted above, in some embodiments, a section of a sensitive film can be selectively treated with one or more ions and/or heat treatment. The following section begins by providing a general description of such treatments, and then provides additional detail as to each of these options (which can be combined in some embodiments).

In some embodiments, the method outlined in FIG. 3A can be further modified by treatment of the sensitive film, as shown in FIG. 3B. As shown in FIG. 3B, the sensitive film 300 can be exposed to a treatment 320 (such as heat and/or ion exposure). In some embodiments, one or more treatments (321, 322, 323, 324, and 325) can be applied to separate portions of the sensitive film 300, such that separately treated portions (301, 302, 303, 304, and 305, in FIG. 3C) are provided. In some embodiments, the treatments can differ (for example, different temperatures, different ions, different concentrations, different durations, etc.), such that one or more of the treated portions (301, 302, 303, 304, 305) will have a different physical/chemical arrangement and thus, interact with one or more targets differently.

In some embodiments, each of the treatments can be applied at a different time. In some embodiments, the various treatments can be applied at different times. In some embodiments, the various treatments can be applied at overlapping times.

In some embodiments, there is no “untreated” portion between the various treated portions. In some embodiments, there is a buffer of untreated sensitive film between each of the treated portions. In some embodiments, there is a buffer of a specific type of treated portion between other portions, so as to act as a known control and/or isolate one portion that is over a vibration detecting unit from another portion that is over a different vibration detecting unit.

In some embodiments, the vibration detecting unit is located on a substrate, and the substrate includes a second vibration detecting unit. The first portion of the sensitive film is positioned over the first vibration detecting unit and the second portion of the sensitive film is positioned over the second vibration detecting unit. Similarly, the other portions can also be positioned over corresponding vibration detecting units. In some embodiments, the treatments are applied to areas of the sensitive film that are placed over the vibration detecting units. In some embodiments, the treatments are applied randomly (or without regard to the position of the vibration detecting units) and the sensitivity of a portion of a film over a vibration detecting unit is determined after the production of the film.

In some embodiments, a graded chemical sensor 380 is provided (see, for example, FIG. 3D. The sensor 380 can include a substrate 381, at least a first vibration detecting unit 351, and a graded sensitive film 300 over both at least a part of the substrate 381 and the vibration detecting unit 351. As noted herein, the graded sensitive film can include a first portion 301 that includes a first set of characteristics that are a same as a portion of a sensitive film that has been heat (and/or ion) treated to a first amount. The sensor can also include a second portion 302 that includes a second set of characteristics that are a same as a portion of a sensitive film that has been heat (or ion) treated to a second amount. In some embodiments, the first amount and the second amount are different. In some embodiments, the second portion 302 can be associated with a second vibration detecting unit 352. In some embodiments, the sensor can include more than two portions (for example see portions 303, 304, and 305 associated with vibration detecting units 353, 354, and 355 in FIG. 3D).

The terms “gradient” or “graded” are used herein to denote a difference that results from a different treatment of a first portion and a second portion. The portions need not abut one another.

In some embodiments, the first portion in the sensor has a characteristic that is different from the second portion (as noted herein). In some embodiments, the characteristic is selected from at least one of: a physical property, a chemical property, or an electrical property. In some embodiments, the first portion has a characteristic that is different from the second portion (or subsequent portions). In some embodiments, the characteristic is selected from at least one of: polarity, dielectric constant, a solubility parameter, hydrophobicity, hydrophilicity, electrical charge, free surface energy, magnetization, magnetic permeability, pH, or conductivity.

In some embodiments, the graded sensitive film includes at least one of titanium oxide, tungsten oxide, or zinc oxide.

As shown in FIG. 3D, in some embodiments, the first portion 301 is positioned over the first vibration detecting unit 351 and the second portion 302 is positioned over a second vibration detecting unit 352. In some embodiments, all of a portion is positioned over a single vibration detecting unit. In some embodiments, only part of the portion is positioned over a single vibration detecting unit. In some embodiments, one or more portions can be positioned over a single vibration detecting unit. In some embodiments, multiple portions are positioned over one or more vibration detecting units.

In some embodiments, the first vibration detecting unit includes a quartz crystal microbalance including a convex shape or an inverse mesa shape.

In some embodiments, the first vibration detecting unit is one of an array of vibration detecting units.

In some embodiments, a method of detecting a presence or absence of a target is provided. In some embodiments, the method involves using any of the treated sensitive films provided herein. In some embodiments, the method includes providing a graded chemical sensor. The graded chemical sensor can include a substrate, a first vibration detecting unit on the substrate, and at least one of: a) a graded sensitive film over both at least a part of the substrate and the first vibration detecting unit, wherein the graded sensitive film includes a first portion that includes a first ion concentration, and a second portion that includes a second ion concentration, and wherein the second ion concentration is different than the first ion concentration; or b) a graded sensitive film over both at least a part of the substrate and the first vibration detecting unit, wherein the graded sensitive film includes a first portion that includes a first set of characteristics that are a same as a portion of a sensitive film that has been heat treated to a first amount, and a second portion that includes a second set of characteristics that are a same as a portion of a sensitive film that has been heat treated to a second amount, wherein the first amount and the second amount are different. The method can further include contacting a sample to the graded sensitive film. If the sample includes the target, the target associates with the sensitive film and changes the vibrational frequency of the sensitive film. The method can further include detecting whether the vibrational frequency of the sensitive film changes (see for example FIG. 2).

In some embodiments, the sensitive films provided herein can be screened against a variety of candidate targets, to see which targets bind to which of the treated portions. This process can allow one to rapidly identify and/or prepare sensitive films that bind to specific targets. In some embodiments, the sensitive film material is selected so that the target is soluble in the film.

In some embodiments, a method of making a graded chemical sensor is provided. In some embodiments, the method can include providing at least a first vibration detecting unit, providing a sensitive film over the first vibration detecting unit, and differentially implanting a first ion concentration in a first portion of the sensitive film relative to a second portion of the sensitive film, thereby providing a graded sensitive film for a graded chemical sensor. In some embodiments, only a portion of the sensitive film is treated. In some embodiments, the entire film is treated. In some embodiments, the entire film can be treated in the same manner.

In some embodiments, the method can include implanting a second ion concentration in the second portion of the sensitive film. The second ion concentration can be different than the first ion concentration. In some embodiments, this can occur more than twice, for example, 3, 4, 5, 6, 7, 8, 9, 10, 20, 30, 40, 50, 60, 70, 80, 90, 100, 200, 300, 400, 500, 600, 700, 800, 900, 1000, 10,000, 100,000, 1,000,000 or more (including any range above any one of the preceding values and any range defined between any two of the preceding values) treatments can be applied to a sensitive film.

In some embodiments, an ion concentration is varied continuously from the first portion to the second portion. In some embodiments, the ion concentration is varied discontinuously from the first portion to the second portion. Thus, a space between the treated portions need not include a gradient in some embodiments. Furthermore, in some embodiments, the concentrations applied (and resulting film) need not be continuous across the surface of the film. In some embodiments, no gradient need be present, and the entire sensitive film is treated.

In some embodiments, differentially implanting can be achieved by applying an ion beam to the sensitive film. In some embodiments, the method can include irradiation with plasma in addition to the ion beam irradiation, and/or it can be applied to the fluid. In some embodiments, differentially implanting includes applying a plasma or fluid across the sensitive film's surface, wherein the plasma or fluid includes the molecules to be used for modifying the sensitive film. When the fluid is applied, one can diffuse the depth and/or direction of the film ions in the fluid in the subsequent heat treatment. In some embodiments, it is possible to exert a predetermined function via the heat treatment.

While not intending to be limited by theory, in some embodiments, implanting the ions introduces a lattice defect in the sensitive film. Thus, in some embodiments, provided herein are sensitive films that include a lattice defect. In some embodiments, the ion treated sensitive film will include more lattice defects than a sensitive film that has not been exposed to the ion treatment.

In some embodiments, differentially implanting ions includes a combinatorial ion implantation technique.

In some embodiments, the film can be heat treated after the ion treatment. This can be done (as noted herein) to provide further differentiation of various portions. In some embodiments, this can be done to fix various properties to various portions of the sensitive film.

In some embodiments, a graded chemical sensor is provided. The sensor can include a substrate, at least one vibration detecting unit, and a graded sensitive film over the substrate. The graded sensitive film can be produced by differentially implanting a first ion concentration in a first portion of the sensitive film relative to a second portion of the sensitive film, thereby providing a graded sensitive film for a graded chemical sensor.

In some embodiments, the first portion of the sensitive film has a first set of characteristics that is different from the second portion of the sensitive film. Similarly, in some embodiments, the other portions can be different from the first and/or one another, for example, 2, 3, 4, 5, 6, 7, 8, 9, 10, 20, 30, 40, 50, 60, 70, 80, 90, 100, 200, 300, 400, 500, 600, 700, 800, 900, 1000, 10,000, 100,000, 1,000,000, or more (including any range above any one of the preceding values and any range defined between any two of the preceding values) of the portions can be different from the first and/or one another.

In some embodiments, the first ion concentration includes at least one of: boron, phosphate, argon or nitrogen.

In some embodiments, the ion treated sensitive film can be part of a graded chemical sensor. The graded chemical sensor can include a substrate, a first vibration detecting unit on the substrate, and a graded sensitive film over both at least a part of the substrate and the first vibration detecting unit. The graded sensitive film can include a first portion that includes a first ion concentration and a second portion that includes a second ion concentration. The second ion concentration can be different than the first ion concentration. Similarly, in some embodiments, the other portions can be different from the first and/or one another, for example, 2, 3, 4, 5, 6, 7, 8, 9, 10, 20, 30, 40, 50, 60, 70, 80, 90, 100, 200, 300, 400, 500, 600, 700, 800, 900, 1000, 10,000, 100,000, 1,000,000, or more (including any range above any one of the preceding values and any range defined between any two of the preceding values) of the portions can be different from the first and/or one another. In some embodiments, the graded chemical sensor can have a portion of its sensitive film that has been heat treated. Thus, in some embodiments, the sensitive film is configured such that it assumes the structure from a heat treatment.

In some embodiments, the graded sensitive film includes at least one of titanium oxide, tungsten oxide, or zinc oxide.

As noted above, in some embodiments, the first portion can be positioned over the first vibration detecting unit and the second portion can be positioned over a second vibration detecting unit. This arrangement can be continued for additional vibration detecting units and/or portions; however, the arrangement is not required for all embodiments.

In some embodiments, the first vibration detecting unit includes a quartz crystal microbalance that includes a convex shape or an inverse mesa shape. In some embodiments, the first vibration detecting unit is one of an array of vibration detecting units.

In some embodiments, a method of making a graded chemical sensor is provided. The method can include providing at least a first vibration detecting unit, providing a sensitive film over vibration detecting unit and differentially heating a first portion of the sensitive film relative to a second portion of the sensitive film, thereby providing a graded sensitive film for a graded chemical sensor.

In some embodiments, the first portion of the sensitive film is heated under a first set of conditions. In some embodiments, the method further includes heating the second portion of the sensitive film. In some embodiments, the method includes heating the second portion of the sensitive film under a second set of conditions. In some embodiments, the second set of conditions is different than the first set of conditions. In some embodiments, this can be repeated any number of times, for example, 2, 3, 4, 5, 6, 7, 8, 9, 10, 20, 30, 40, 50, 60, 70, 80, 90, 100, 200, 300, 400, 500, 600, 700, 800, 900, 1000, 10,000, 100,000, 1,000,000, or more (including any range above any one of the preceding values and any range defined between any two of the preceding values) times to create various heat treated portions. In some embodiments, the entire sensitive film is heat treated in a same manner.

In some embodiments, a condition of heating is varied continuously from the first portion to the second portion. In some embodiments, the condition of heating is varied discontinuously from the first portion to the second portion. Thus, heat need not be applied to create continuous gradient and the film need not have a continuous gradient in it.

While not intending to be limited by theory, in some embodiments, differentially heating promotes at least one of a reaction or an atomic reordering thereby creating a heat stabilized portion of the sensitive film. Thus, in some embodiments, the heat treated films can include a portion that has greater stabilization and/or contains fewer initial reactants from the film as more of the materials in the film have more completely reacted.

In some embodiments, differentially heating includes differentially irradiating a first portion of the sensitive film relative to a second portion of the sensitive film. In some embodiments, this can be repeated any number of times, for example, 2, 3, 4, 5, 6, 7, 8, 9, 10, 20, 30, 40, 50, 60, 70, 80, 90, 100, 200, 300, 400, 500, 600, 700, 800, 900, 1000, 10,000, 100,000, 1,000,000, or more times to create various heat treated portions (including any range above any one of the preceding values and any range defined between any two of the preceding values).

In some embodiments, differentially heating alters at least one characteristic of the differentially heated portion of the sensitive film. In some embodiments, the at least one characteristic includes at least one of: a polarity, a dielectric constant, a solubility parameter, hydrophobicity, hydrophilicity, electrical charge, or conductivity.

In some embodiments, the first vibration detecting unit is located on a substrate. The substrate can include a second vibration detecting unit. The first portion of the sensitive film can be positioned over the first vibration detecting unit and the second portion of the sensitive film can be positioned over the second vibration detecting unit.

In some embodiments, a graded chemical sensor is provided. The graded chemical sensor includes a substrate, at least one vibration detecting unit, and a graded sensitive film over both at least part of the substrate and the at least one vibration detecting unit. The graded sensitive film being produced by differentially heat treating a first portion of the sensitive film relative to a second portion of the sensitive film. In some embodiments, the first portion of the sensitive film has a first set of characteristics that is different from the second portion of the sensitive film.

In some embodiments, the above sensitive films, devices and/or arrays are sized appropriately for use in a mobile device. In some embodiments, the application of a graded sensitive film allows one to avoid and/or minimize degraded detection precision and degraded reliability. In some embodiments, the array or device can be used to detect odors. In some embodiments, the array or device can be used to detect flatus. In some embodiments, the film layers are not formed by dipping. In some embodiments, the layers are not formed by spraying. In some embodiments, the sensitive film and/or device and/or array is part of a mobile device, such as a phone, laptop, breathalyzer, security wands, watch, tablet, PDA, glasses, head mounted displays and/or handheld device. In some embodiments, the device is part of a healthcare kit or medical device.

The embodiments herein present arrays, sensitive films and devices that can be employed within devices for checking fluids such as liquids and gases for various targets. These devices can have a wide range of applications including environmental chemical monitoring, industrial process control, leakage tests, automobile discharge gas tests, disease diagnosis and health management, quality control through monitoring of food and drinking water, and military purposes such as detection of weapons or explosives.

In some embodiments, the graded sensitive film embodiments provided herein do not suffer from a reduced specific surface area for the sensitive portion. As such, unlike in other technologies, the signal intensity need not become weaker upon its use in a mobile device.

As will be appreciated from the disclosure herein, in some embodiments, one or more of the devices and methods provided herein can provide any number of advantages. In some embodiments, the vibration-detecting array allows for miniaturizing a chemical sensor array. This can be achieved by a graded synthetic film, which can be formed on a quartz substrate by establishing a distribution of synthesis parameters (control of physical properties) of the sensitive films. Such parameters can include the heat-treatment history and/or ion implantation concentration. These can be done on a convex QCM (having a thick portion on the surface) or a reverse-mesa QCM (produced by engraving the surface into a concave shape). In some embodiments, this allows for one or more of the following advantages: the reduction in interference, such as propagation or reflection, between crystal oscillators arranged in an array; detection sites that are easily arranged in an array; reduction in variation in deposition across the elements; graded synthetic films can be formed on all vibration-detecting portions at one time; and/or allowing for an efficient search for providing a sensitive film appropriate for a particular odor. In some embodiments, the above-described results provide for chemical sensors featuring high-accuracy and high-reliability detection that can be made small enough to fit in a mobile device and can also be produced at low cost.

In some embodiments, the film can include a titanium oxide, a zinc oxide, or both a zinc oxide and a titanium oxide. The electrical characteristics such as the resistance and/or conductivity of such materials can be changed by ion implantation to enhance the sensor sensitivity. Thus, an arrayed chemical sensor on which ion-implanted graded film is formed by this technique can be used to create a sensitive film with a wide variety of properties. In some embodiments, the sensitive film can be used to efficiently search for sensitive films for the detection of various target molecules, as these treatments (heat or ion implantation) allows for a very wide variety of properties to be provided within a relatively small area on the sensitive film.

In some embodiments, the treatment approaches provided herein allow one to deposit sensitive films onto oscillators in the array such that their physical properties are differentiated from one another. In some embodiments, the treatment approaches provided herein allow one to reduce interference between oscillators. In some embodiments, the treatment approaches provided herein allow one to reduce the variation in the amounts of deposited sensitive films. In some embodiments, the treatment approaches provided herein allow one to reduce the burden and time to prepare a wide variety of solutions and accordingly deposit them onto the oscillators. In some embodiments, the treatment approaches provided herein allow one to find (or develop) a sensitive film appropriate for a particular odor.

### Example 1

### Producing a Temperature-Graded Film

A sensitive film including equal parts tungsten oxide and zinc oxide is deposited onto a single quartz substrate having 25 vibration-detecting portions using a sputtering system. A heat-treated graded synthetic film is formed under 25 different heat treatment conditions to provide a five by five array of detection sites (as shown in FIG. 3C). The heat is applied via laser light during the film deposition through the rear surface of the square film-deposition substrate. The intensity of the laser light is varied across each of the five rows.

The duration of irradiation is varied across each of the five columns (each progressive column being exposed to 30 seconds more light), thereby endowing the sensitive film with 25 different heat-treatment histories, one for each area in the sensitive film over each vibration detection unit.

### Example 2

### Producing an Ion-Implanted Graded Film

A sensitive film made of titanium oxide is deposited onto a quartz substrate having 100 vibration-detecting units arranged in a 10 by 10 grid. The deposition is performed by chemical vapor deposition.

An ion-implanted graded film is formed in the sensitive film under 100 different conditions to produce 100 different detection sites (each of which is positioned over one of the vibration detecting units).

The sensitive film is implanted with varying levels of argon ions in the X direction of the grid and varying levels of nitrogen ions in the Y direction of the grid. The scanning speed of the ion beam is controlled in the X direction, and the movable mask is controlled in the Y direction. The duration of exposure of each treated area to the ions increases by 10 seconds for each progressive portion (thus, 10, 20, 30, 40, 50, 60, 70, 80, 90, and 100 seconds for the full row or column. The ions are applied by the combinatorial ion implantation technique.

The combinatorial library produced with this system will have 10×10 pixels for the 100 vibration detection units. In the alternative, this can also be performed to provide 100×100 area (over 100×100 vibration detection units).

### Example 3

### Seven-Array Crystal Oscillator Substrate Having Ternary Composition-Graded Films Deposited Thereon

A substrate can be formed as outlined below to provide for a seven-array QCM substrate (a triangle with 16 mm sides) having separated electrodes on one side thereof. The final film can include ZrO₂—WO₃—TiO2. The film deposition system is a CMS-6400 combinatorial film deposition system.

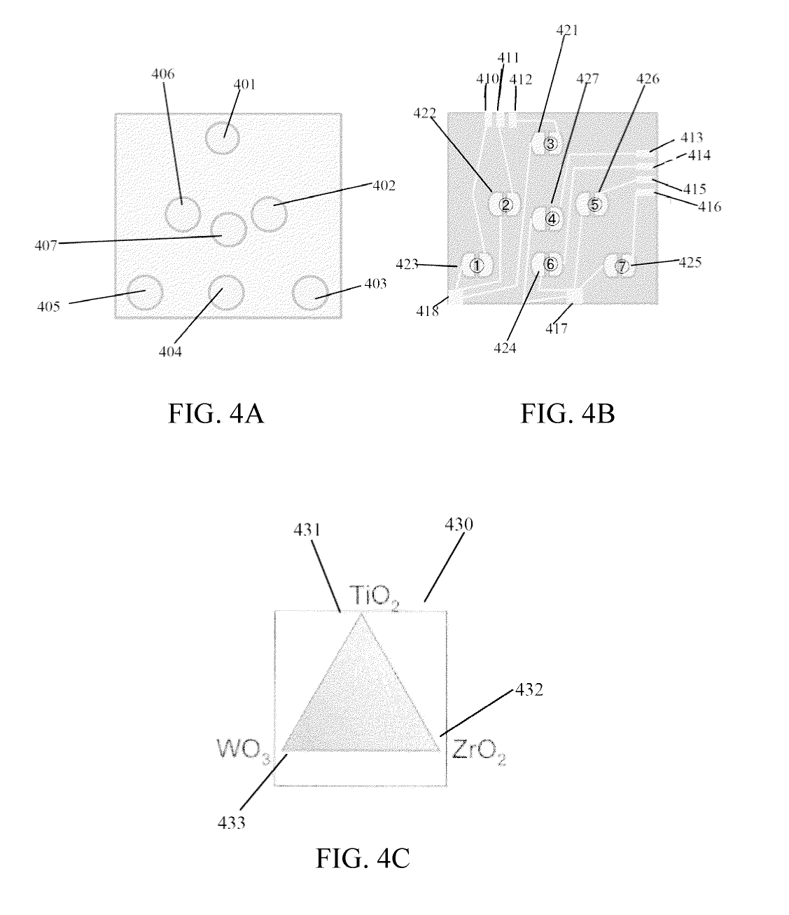

The film is deposited in a triangular area (16 mm on each side) including 7 electrode portions on a square quartz substrate with sides of 16 mm (see FIG. 4A for the front of the vibration detection units 401, 402, 403, 404, 405, 407, and 406 and FIG. 4B for the back of the electrodes 425, 424, 423, 422, 421, 427, and 426). Three constituent layers, each measuring 5 nm, are deposited in the triangular area in as many as 20 layers. The deposition conditions can be as shown in Table 1.

TABLE 1

| | |
|---|---|
| Target | 50.8 mm in dia. with 3N for each of ZrO2, |
| | WO3, and TiO2 |
| Ultimate pressure | <1 * 10⁵ Pa |
| Gas pressure/flow volume | Ar 0.7 Pa, 50 sccm |
| Power applied | ZrO2 = 200 W, WO3 = 200 W, TiO2 = |
| | 250 W |
| Deposition temperature | Room Temperature |

The parameters for the deposition are shown in Table 2 (for a 5-nm film).

TABLE 2

| | | | 5 nm film deposition |
|---|---|---|---|
| Target | Power | Rate (nm/s) | time |
| ZrO2 | 200 W | 0.036 | 138.8 s |
| WO3 | 200 W | 0.063 | 79.2 s |
| TiO2 | 250 W | 0.0173 | 288.4 s |

This results in a sensitive film 430 that includes WO₃ (at 433), TiO₂ (at 431), and ZrO₂ (at 432) as shown in FIG. 4C. This graded film can then further be employed in, for example, the method of Example 1 or Example 2, to provide for a system that is varied not only in film composition between the three deposited materials, but also further varied by heat treatment and/or ion implantation treatment.

In some embodiments, the various electrodes (425, 424, 423, 422, 421, 427, and 426) can be in electrical communication with an electrical lead, such as 410, 411, 412, 413, 414, 415, and 416. In some embodiments, the electrodes can share a first common lead 418 and/or a second common lead 417.

### Example 4

### Use of a Sensor Device with Air Sample

The sensor device of Example 1 (or in the alternative Example 2) is provided and activated through the use of an electrical current. A baseline signal from the device is observed in the absence of any molecules in the environment that would otherwise bind to the sensitive film.

A sample of air to be tested is blown onto a surface of the sensitive film. The presence of hydrogen sulfide in the sample of air will associate with the ZrO₂ film, changing the mass of the film, and altering the frequency of vibration of the film. This change in vibration is detected by the vibration detecting unit, and transmitted to a computer, or in the alternative, a display device, where the change in signal can be observed, thereby demonstrating the detection of the presence of hydrogen sulfide in the sample of air.

The presence of either a convex vibration detecting unit or an inverse mesa detection unit allows for a reduction in possible interference that could otherwise occur in the array.

### Example 5

### Determination of Targets

The sensor device of Example 1 (or in the alternative Example 2) is provided and activated through the use of an electrical current. A baseline signal from the device is observed under nitrogen to provide a baseline measurement.

A sample of air containing carbon monoxide is blown onto a surface of the sensitive film. The sensor device is monitored to see if the presence of carbon monoxide results in a change in signal from the device. If there is a change in signal, the vibration detecting unit that indicates a sensitivity to carbon monoxide is noted so that when a signal comes from that vibration detecting unit in the future, it can be correlated to the presence of carbon monoxide. If there is no change in signal, the sensitive film can go through a subsequent round of treatment (thereby altering the net treatment parameters further) and the testing process can be repeated to see if the further treated sensitive film displays any sensitivity to carbon monoxide.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A method of making a graded chemical sensor, the method comprising:
providing at least a first vibration detecting unit;
providing a sensitive film formed over a substrate and the first vibration detecting unit; and
differentially treating a first portion of the sensitive film relative to a second portion of the sensitive film, each of the first and second portions of the sensitive film exhibiting substantially the same material composition;
wherein differentially treating the first portion of the sensitive film relative to the second portion of the sensitive film includes at least one of:
differentially implanting at least one of a first ion concentration or a first type of ion in the first portion of the sensitive film relative to the second portion of the sensitive film; or
differentially heating the first portion of the sensitive film relative to the second portion of the sensitive film.

2. The method of claim 1, further comprising treating the second portion of the sensitive film, wherein treating the second portion of the sensitive film includes at least one of:
implanting a second ion concentration or a second type of ion in the second portion of the sensitive film, the second ion concentration or the second ion type is different than the first ion concentration or the first ion type; or
heating the second portion of the sensitive film, wherein the first portion of the sensitive film is heated under a first set of conditions and the second portion of the sensitive film is heated under a second set of conditions different than the first set of conditions.

3. The method of claim 2, wherein differentially treating the first portion of the sensitive film relative to the second portion of the sensitive film includes continuously varying at least one of an ion concentration or a heat treatment from the first portion to the second portion.

4. The method of claim 2, wherein differentially treating the first portion of the sensitive film relative to the second portion of the sensitive film includes discontinuously varying at least one of an ion concentration or a heat treatment from the first portion to the second portion.

5. The method of claim 1, wherein differentially treating comprises at least one of:
applying an ion beam to the sensitive film; or
irradiating the sensitive film.

6. The method of claim 1, wherein differentially treating at least one of introduces a lattice defect in the sensitive film, promotes a reaction, or promotes atomic reordering.

7. The method of claim 1, wherein differentially implanting comprises a combinatorial ion implantation technique.

8. (canceled)

9. The method of claim 1, wherein the first portion of the sensitive film is over the first vibration detecting unit and wherein the second portion of the sensitive film is over a second vibration detecting unit.

10.-17. (canceled)

18. The method of claim 1, wherein differentially treating the first portion of the sensitive film relative to the second portion of the sensitive film alters at least one characteristic of the first portion of the sensitive film, the at least one characteristic including at least one a polarity, a dielectric constant, a solubility parameter, hydrophobicity, hydrophobicity, electrical charge or conductivity.

19.-20. (canceled)

21. A graded chemical sensor, the sensor comprising:
a substrate;
at least one vibration detecting unit; and
a graded sensitive film over the substrate, the graded sensitive film including a first portion and a second portion;
wherein the graded sensitive film, including the first and second portions thereof, is substantially continuous;
wherein at least one of:
the first portion exhibits at least one first ion characteristic that is different than at least one second ion characteristic exhibited by the second portion; or
the first portion exhibits at least one first physical characteristic different than at least one second physical characteristic exhibited by the second portion, the at least one first physical characteristic including at least one of: a polarity, a dielectric constant, a solubility parameter, hydrophobicity, hydrophilicity, electrical charge, or conductivity.

22. (canceled)

23. The graded chemical sensor of claim 21, wherein the at least one first ion characteristic comprises a first ion type including at least one of: boron, phosphate, argon, or nitrogen.

24.-25. (canceled)

26. The graded chemical sensor of claim 21, wherein
the graded sensitive film is positioned over both at least a part of the substrate and the at least one vibration detecting unit.

27. (canceled)

28. The graded chemical sensor of claim 21, wherein the at least one first physical characteristic is imparted to the first portion by heat treatment thereof.

29. The graded chemical sensor of claim 21, wherein the first portion includes a heat treated portion.

30. The graded chemical sensor of claim 21, wherein the graded sensitive film comprises at least one of titanium oxide or zinc oxide.

31. The graded chemical sensor of claim 21, wherein the at least one vibration detecting unit includes a first vibration detecting unit and a second vibration detecting unit, the first portion positioned over the first vibration detecting unit and the second portion positioned over the second vibration detecting unit.

32. The graded chemical sensor of claim 31, wherein the first vibration detecting unit comprises a quartz crystal microbalance comprising a convex shape or an inverse mesa shape.

33. The graded chemical sensor of claim 31, wherein the first vibration detecting unit includes an array of vibration detecting units.

34.-40. (canceled)

41. A method of detecting a presence or absence of a target, the method comprising:
providing a graded chemical sensor, the graded chemical sensor comprising:
a substrate;
a first vibration detecting unit on the substrate; and
a graded sensitive film over the substrate, the graded sensitive film including a first portion and a second portion;
wherein the graded sensitive film, including the first and second portions thereof, is substantially continuous;
wherein at least one of:
the first portion exhibits at least one first ion characteristic that is different than at least one second ion characteristic exhibited by the second portion; or
the first portion exhibits at least one first physical characteristic different than at least one second physical characteristic exhibited by the second portion, the at least one first physical characteristic including at least one of: a polarity, a dielectric constant, a solubility parameter, hydrophobicity, hydrophilicity, electrical charge, or conductivity;
contacting a sample to the graded sensitive film, wherein if the sample comprises the target, the target associates with the sensitive film and changes the vibrational frequency of the sensitive film; and
detecting whether the vibrational frequency of the sensitive film changes.

42. The method of claim 1, wherein the sensitive film, including the first and second portions thereof, is substantially continuous.

43. The method of claim 1, wherein the first and second portions of the sensitive film exhibit the same material composition after the act of differentially treating.

44. The method of claim 1, wherein differentially heating the first portion of the selective film relative to the second portion of the sensitive film includes heating the first portion of the sensitive film in a non-contact manner.

45. The graded chemical sensor of claim 21, wherein the at least one first ion characteristic includes at least one of a first type of ion or a first ion concentration, and wherein the at least one second ion characteristic includes at least one of a second type of ion different than the first type of ion or a second ion concentration different than the first ion concentration.

46. The method of claim 41, wherein the at least one first ion characteristic includes at least one of a first type of ion or a first ion concentration, and wherein the at least one second ion characteristic includes at least one of a second type of ion different than the first type of ion or a second ion concentration different than the first ion concentration.
